(19) United States
(12) Patent Application Publication
(10) Pub. No.: US 2021/0369665 A1
(43) Pub. Date: Dec. 2, 2021

(54) METHODS OF TREATING VIRAL INFECTION USING PROTEASE INHIBITORS

(71) Applicant: Lowell Therapeutics, Inc., Lowell, MA (US)

(72) Inventors: James A. Wilkie, Dunstable, MA (US); Lakhmir Chawla, San Diego, CA (US); Peter Jordan, Haymarket, VA (US); Elaine C. Liong, Del Mar, CA (US); Michelle M. Merrigan, San Diego, CA (US); Martha C. Farmer, Manchester, MA (US)

(21) Appl. No.: 17/330,031

(22) Filed: May 25, 2021

Related U.S. Application Data
(60) Provisional application No. 63/030,011, filed on May 26, 2020.

(51) Int. Cl. A61K 31/245; A61K 45/06; A61K 38/57

(52) U.S. Cl. CPC A61K 31/245; A61K 38/57; A61K 45/06

(57) ABSTRACT

The present disclosure provides methods of treating a viral infection comprising administering a protease inhibitor, optionally conjointly with an anticoagulant therapy and/or antiviral agent.

### RELATED APPLICATIONS

This application claims the benefit of U.S. Provisional Application No. 63/030,011, filed on May 26, 2020.

### BACKGROUND

The recent and ongoing pandemic of novel Coronavirus (also known as SARS-CoV-2, 2019-nCoV, or COVID-19) is resulting in respiratory viral infections, inflammatory lung injury, and death. While similar to previous Coronavirus epidemics, such as severe acute respiratory syndrome (SARS-CoV) and Middle East Respiratory Syndrome (MERS-CoV), which have higher fatality rates, SARS-CoV-2 appears to be more infectious and, as a result, overall number of deaths from COVID-19 are much greater than that of either SARS or MERS. As of May 17, 2021, SARS-CoV-2 has infected approximately 163 million people with an estimated 3.3 million deaths worldwide. Patients who develop COVID-19 are provided medical care intended to relieve symptoms and, for severe cases, are provided support for vital organ function.

Currently, information is emerging for COVID-19 patients with physicians observing systemic clotting in the severe and critically ill patients (disseminated intravascular coagulation) with clots showing up in the lungs, kidneys, liver, and heart. D-dimer (a fibrin degradation product indicating thrombosis) is linked to higher odds of death in the hospital. Coronavirus infections are presenting a comorbidity of disseminated intravascular coagulation referred to as COVID-19-associated coagulopathy (CAC), where seriously ill patients are showing elevated fibrinogen and D-dimer levels (3-4 fold) related to coagulation activation from infection/sepsis, cytokine storm and impending organ failure. Clinical professionals are now providing interim guidance for the use of blood thinners (anticoagulants) for potential treatment of the comorbidities associated with COVID-19. As these patients are meeting the criteria for disseminated intravascular coagulation (DIC), they may be treated with anticoagulants like heparin or nafamostat which is approved for use as an anticoagulant in DIC in Japan.

Methods for effectively managing the infection and the inflammation cytokine response are still to be determined as the relevant information on patient biomarkers and characteristics is developing daily. Improved methods for managing the symptoms of COVID-19 are needed to reduce the demand for ventilators and other equipment, the length of hospitalizations, and the number of deaths resulting from these infections.

### SUMMARY OF THE INVENTION

Protease inhibitors, which are currently used for treating cancer, are also beneficial for the treatment of coronavirus and other viral infections. The mode of action of some protease inhibitors relies on serine protease inhibition, which reduces the opportunity for viral entry into cells while alleviating the impact of inflammation and edema associated with viral illness.

One such serine protease inhibitor is nafamostat. Appropriate doses of nafamostat to treat COVID-19 are unknown. This is compounded by the fact that patients with SARS-CoV-2 infection who develop COVID-19 are routinely treated with anticoagulants to prevent clotting (e.g., venous thromboembolism [VTE] prophylaxis). Since these anticoagulants can interact with the anticoagulation effects of nafamostat, the correct dose of nafamostat that is utilized in conjunction with other anticoagulants is critical to understand for both safety and efficacy.

In certain aspects, methods are disclosed herein to treat a viral infection, comprising administering a protease inhibitor at a starting dose of 5-200 mcg/kg/hour over 2 hours. In these methods, the protease inhibitor is optionally administered conjointly with an anticoagulation therapy and/or antiviral agent.

In certain aspects, methods are disclosed herein to treat a viral infection, comprising administering a protease inhibitor at a maintenance dose of 10-700 mcg/kg/hour for up to 21 days. In these methods, the protease inhibitor is optionally administered conjointly with an anticoagulation therapy and/or antiviral agent.

In certain aspects, methods are disclosed herein to treat hypoxemia, pulmonary intravascular coagulopathy, disseminated intravascular coagulation, viremia, septic shock, systemic inflammatory response syndrome (SIRS), neurovascular ischemia, cardiovascular ischemia, acute respiratory distress syndrome (ARDS), edema, stroke, and/or vascular leak syndrome, comprising administering a protease inhibitor at a starting dose of 5-200 mcg/kg/hour for over 2 hours or a maintenance dose of 10-700 mcg/kg/hour for up to 21 days.

### DETAILED DESCRIPTION OF THE INVENTION

### General

Disclosed herein are methods of treating a viral infection by administering a protease inhibitor to a subject with the intent of inhibiting viral propagation and infectivity. Because coronaviruses rely on proteolysis of relevant viral proteins for entry, inhibition of the proteases will result in minimizing viral replication and thereby control inflammation, control vascular leak syndrome, and cause disaggregation of platelets and anticoagulation of blood.

The methods disclosed herein determine the type and level of anticoagulant dose (e.g., VTE prophylaxis standard dose with heparin) that pairs with a dose of a protease inhibitor for initiation and maintenance treatment. For example, the invention discloses a starting dose and dosing period of protease inhibitor nafamostat (10-100 mcg/kg/hour, over 2 hours) for use in patients with COVID-19 who are also treated conjointly with heparin at intermediate anticoagulant dose (>20,000 units per day and activated partial thromboplastin time (aPTT) less than 60 seconds). Similarly, the invention discloses a maintenance dose and dosing period of nafamostat (1-500 mcg/kg/hour, up to 7 days) in patients with COVID-19 who are also conjointly treated with heparin at full anticoagulant dose (>20,000 units per day and aPTT greater than 60 seconds).

In addition, disclosed herein are methods of treating hematological and pulmonary conditions with a protease inhibitor. The conditions include hypoxemia, pulmonary intravascular coagulopathy, disseminated intravascular coagulation, viremia, septic shock, systemic inflammatory response syndrome (SIRS), neurovascular ischemia, cardiovascular ischemia, acute respiratory distress syndrome (ARDS), edema, stroke and/or vascular leak syndrome. The method may comprise selecting a subject, or a subpopulation of subjects, to determine for whom a protease inhibitor agent is suitable based on selection criteria. For example, vascular leak syndrome is characterized by elevated levels of C-reactive protein (CRP) and IL-6 biomarkers. A subject with elevated levels of these biomarkers may be selected for treatment with a protease inhibitor.

### Definitions

As used in this specification, “a” and “an” can mean one or more. As used herein in the claim(s), when used in conjunction with the word “comprising,” the words “a” and “an” can mean one or more than one. As used herein, “another” can mean at least a second or more.

As used herein, “anticoagulating” includes inhibiting or reducing the coagulation of blood. For example, an agent anticoagulates blood if the blood has a longer clotting time in its presence as compared to in its absence.

As used herein, “without substantially affecting anticoagulation” refers to a change (or lack thereof) in a blood coagulation parameter such as activated partial thromboplastin time (aPTT), prothrombin time (PT), international normalized ratio (PT/INR), thromboelastography (TEG), or the activated coagulation time (ACT) that is at most 50% (e.g., −50%, −25%, −15%, −10%, −5%, 0%, +5%, +10%, +15%, +25%, +50%). For example, if the systemic ACT of a subject changes from 100 seconds to 110 seconds during a procedure, the procedure does not substantially affect anticoagulation in the subject, since the change in ACT is +10%, which is not more than 50%.

As used herein, “systemic anticoagulation” refers to anticoagulation within a subject's body, which, for example, can be measured from blood drawn directly from the patient. Various coagulation parameter values can be used as a measure of systemic anticoagulation, such as ACT, aPTT, or a combination thereof. In some embodiments, a therapeutically effective change in a coagulation parameter from blood drawn directly from the patient indicates a therapeutically effective systemic anticoagulation.

As used herein, “therapeutically effective rate” includes any rate that leads to an improvement in the treated condition. For example, a rate is therapeutically effective if it leads to curing, relieving, or ameliorating to any extent a symptom of an illness or medical condition or to preventing further worsening of such a symptom.

A “biomarker” can be anything that can be used as an indicator of a particular physiological state of an organism. For example, a biomarker can be a level of an analyte, metabolite, by-product, mRNA, enzyme, peptide, polypeptide, or protein associated with a particular physiological state, criteria, or score (e.g., the International Society on Thrombosis and Hemostasis (ISTH) DIC criteria, the Japanese Association for Acute Medicine (JAAM) DIC criteria, a clinical evaluation score such as a Sequential Organ Failure Assessment (SOFA) or Acute Physiology and Chronic Health Evaluation (APACHE) score).

The term “subject” refers to a mammal, including, but not limited to, a human or non-human mammal, such as a bovine, equine, canine, ovine, or feline. In some embodiments, the subject is a human. In the specification, the term “patient” is used interchangeably with the term “subject.”

The term “binding” or “interacting” refers to an association, which may be a stable association, between two molecules, due to, for example, electrostatic, hydrophobic, ionic and/or hydrogen-bond interactions under physiological conditions.

As used herein, the term “subject” means a human or non-human animal selected for treatment or therapy.

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

In certain embodiments, therapeutic compounds may be used alone or conjointly administered with another type of therapeutic agent (e.g., an immuno-oncology agent or a chemotherapeutic agent disclosed herein). As used herein, the phrase “conjoint administration” refers to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, an individual who receives such treatment can benefit from a combined effect of different therapeutic compounds.

The term “thrombosis treatment drug” means a substance that inhibits aggregation of blood-clotting proteins and cells related thereto such as platelets.

The term “thrombin” is a serine protease having a central role in hemostasis through the conversion of fibrinogen to fibrin.

### Viral Infections and Antiviral Agents

The methods of the invention are useful for treating viral infection. Viruses are small infectious agents which contain a nucleic acid core and a protein coat, but are not independently living organisms. A virus cannot multiply in the absence of a living cell within which it can replicate. Viruses enter specific living cells either by transfer across a membrane or direct injection, and multiply, causing disease. The multiplied virus can then be released and infect additional cells. Some viruses are DNA-containing viruses and others are RNA-containing viruses. The genomic size, composition, and organization of viruses shows tremendous diversity.

In some embodiments, the viral infection may be caused by an arbovirus, adenovirus, alphavirus, arenaviruses, astrovirus, BK virus, bunyaviruses, calicivirus, cercopithecine herpes virus 1, Colorado tick fever virus, coronavirus, Coxsackie virus, Crimean-Congo hemorrhagic fever virus, cytomegalovirus, Dengue virus, ebola virus, echinovirus, echovirus, enterovirus, Epstein-Barr virus, flavivirus, foot-and-mouth disease virus, hantavirus, hepatitis A, hepatitis B, hepatitis C, herpes simplex virus I, herpes simplex virus II, human herpes virus, human immunodeficiency virus type I (HIV-I), human immunodeficiency virus type II (HIV-II), human papillomavirus, human T-cell leukemia virus type I, human T-cell leukemia virus type II, influenza, Japanese encephalitis, JC virus, Junin virus, lentivirus, Machupo virus, Marburg virus, measles virus, mumps virus, naples virus, norovirus, Norwalk virus, orbiviruses, orthomyxovirus, papillomavirus, papovavirus, parainfluenza virus, paramyxovirus, parvovirus, picornaviridae, poliovirus, polyomavirus, poxvirus, rabies virus, reovirus, respiratory syncytial virus, rhinovirus, rotavirus, rubella virus, sapovirus, smallpox, togaviruses, Toscana virus, varicella zoster virus, West Nile virus, Yellow Fever virus, or Zika virus.

In some embodiments, the viral infection may be cause by an enveloped virus. An enveloped virus is an animal virus which possesses a membrane or “envelope,” which is a lipid bilayer containing viral proteins. The envelope proteins of a virus play a pivotal role in its lifecycle. They participate in the assembly of the infectious particle and also play a crucial role in virus entry by binding to a receptor present on the host cell and inducing fusion between the viral envelope and a membrane of the host cell. Enveloped viruses can be either spherical or filamentous (rod-shaped) and include but are not limited to filoviruses, such as Ebola virus or Marburg virus, Arboroviruses such as Togaviruses, flaviviruses (such as hepatitis-C virus), bunyaviruses, and Arenaviruses, Orthomyxoviridae, Paramyxoviridae, poxvirus, herpesvirus, hepadnavirus, Rhabdovirus, Bornavirus, and Arterivirus.

In some embodiments, the viral infection may be caused by influenza A virus, influenza B virus, and influenza C virus. Influenza type A viruses are divided into subtypes based on two proteins on the surface of the virus. These proteins are called hemagglutinin (HA) and neuraminidase (NA). There are 15 different HA subtypes and 9 different NA subtypes. Subtypes of influenza A virus are named according to their HA and NA surface proteins, and many different combinations of HA and NA proteins are possible. For example, an “H7N2 virus” designates an influenza A subtype that has an HA 7 protein and an NA 2 protein. Similarly, an “H5N1” virus has an HA 5 protein and an NA 1 protein. Only some influenza A subtypes (i.e., H1N1, H2N2, and H3N2) are currently in general circulation among humans. Other subtypes such as H5 N1 are found most commonly in other animal species and in a small number of humans, where it is highly pathogenic. For example, H7N7 and H3N8 viruses cause illness in horses. Humans can be infected with influenza types A, B, and C. However, the only subtypes of influenza A virus that normally infect people are influenza A subtypes H1N1, H2N2, and H3N2 and recently, H5N1. The influenza A and B viruses that routinely spread in people (human influenza viruses) are responsible for seasonal flu epidemics each year. It has recently been reported that there is an association between seasonal flu and venous thromboembolism (VTE).

In some embodiments, the viral infection is caused by an arbovirus. Arboviruses are a group of more than 400 enveloped RNA viruses that are transmitted primarily (but not exclusively) by arthropod vectors (mosquitoes, sand-flies, fleas, ticks, lice, etc). Arborviruses have been categorized into four virus families, including the togaviruses, flaviviruses, arenaviruses, and bunyaviruses. Togaviruses includes the genuses Alphavirus (e.g., Sindbis virus, which is characterized by sudden onset of fever, rash, arthralgia or arthritis, lassitude, headache and myalgia) and Rubivirus (e.g., Rubella virus, which causes Rubella in vertebrates). The Flavivirus genus includes yellow fever virus, dengue fever virus, Japanese encaphilitis (JE) virus, and West Nile virus.

Dengue virus is the most common cause of mosquito-borne viral diseases in tropical and subtropical regions around the world, and is expanding in geographic range and also in disease severity. Currently, there are no licensed drugs for the treatment of dengue. The virus is a small, enveloped, icosahedral virus, with positive strand RNA of 11,000 nucleotides. There are four distinct serotypes of dengue that cause similar disease symptoms, serotypes 1-4 (DENV-1, DENV-2, DENV-3, and DENV-4) that co-circulate in many areas of the world and give rise to sequential epidemic outbreaks when the number of susceptible individuals in the local population reaches a critical threshold and weather conditions favor reproduction of the mosquito vectors Aedes aegypti and Aedes albopictus. Dengue virus infection causes a characteristic pathology in humans involving dysregulation of the vascular system. In some patients with dengue hemorrhagic fever (DHF), vascular pathology can become severe, resulting in extensive microvascular permeability and plasma leakage into tissues and organs. Recently, the mast cell-derived proteases, tryptase and chymase, have been implicated in the immune mechanism by which dengue induces vascular pathology and shock.

West Nile virus is one of the most widely distributed flaviviruses in the world and has emerged in recent years to become a serious public health threat. West Nile virus is an enveloped positive-strand RNA virus, with a genome that encodes 3 structural and 7 non-structural proteins as a single polypeptide that then co- and post translationally processed to yield the 10 proteins. The 3 virus structural proteins are the capsid (C) protein, pre-membrane protein (prM) which is cleaved during virus maturation to yield the membrane (M) protein and envelope (E) protein. The E protein contains the receptor binding and fusion functions of the virus. Severe viral infection is characterized by fever, convulsions, muscle weakness, vision loss, numbness, paralysis, and coma. Because West Nile virus is capable of eliciting pathology in the brain, it has been postulated that the virus may modulate blood-barrier vascular permeability.

In some embodiments, the viral infection is caused by a respiratory syncytial virus (RSV). The respiratory syncytial virus (RSV) is an enveloped, negative-sense, single-stranded RNA virus of the genus Pneumovirinae and of the family Paramyxoviridae. Symptoms in adults typically resemble a sinus infection or the common cold, although the infection may be asymptomatic. In older adults (e.g., >60 years), RSV infection may progress to bronchiolitis or pneumonia. Symptoms in children are often more severe, including bronchiolitis and pneumonia. The RNA genome of the RSV virus is approximately 15 kb and encodes 11 viral proteins, which includes the F (fusion) protein that is a transmembrane protein of the virus and the M (matrix) protein that is a core protein of the virus. RSV infections are known to cause vascular complications and the infection has been associated with venous thromboembolism.

In some embodiments, the viral infection is caused by a coronavirus. Coronaviruses are a family of enveloped, positive-sense, single-stranded RNA viruses, that was first described in 1949. These viruses are found in mice, rats, dogs, cats, turkeys, horses, pigs, and cattle. These viruses infect humans, and the pathology of these viruses in humans may vary.

The coronavirus genome, approximately 27-32 Kb in length, is the largest found in any of the RNA viruses. Large Spike (S) glycoproteins protrude from the virus particle giving coronaviruses a distinctive corona-like appearance when visualized by electron microscopy. The virus is further classified into 4 groups: the α, β, γ, and δ CoVs by phylogenetic clustering, of which α and β are known to cause infection in humans. It is believed that the gammacoronavirus and deltacoronavirus genera may infect humans. Non-limiting examples of alphacoronaviruses include human coronavirus 229E (229E-CoV), human coronavirus NL63 (NL63-CoV), porcine epidemic diarrhea virus (PEDV), Canine coronavirus HuPn-2018 (CCoV-HuPn-2018), and Transmissible gastroenteritis coronavirus (TGEV). A non-limiting example of a deltacoronaviruses is the Swine Delta Coronavirus (SDCV). Non-limiting examples of betacoronaviruses include Middle East respiratory syndrome coronavirus (MERS-CoV), Severe Acute Respiratory Syndrome coronavirus (SARS-CoV), Severe Acute Respiratory Syndrome coronavirus 2 (SARS-CoV-2), Human coronavirus HKU1 (HKU1-CoV), Human coronavirus OC43 (OC43-CoV), Murine Hepatitis Virus (MHV-CoV), Bat SARS-like coronavirus WIV1 (WIV1-CoV), and Human coronavirus HKU9 (HKU9-CoV).

Coronaviruses facilitate entry to the cell via host protease activation of viral surface proteins (such as TMPRSS2). These host proteases are shown to increase the infectivity of the virus by a thousand-fold.

Viruses may enter cells through the endosomal pathway. For example, SARS-CoV-2 can use the endosomal pathway, which is reliant on the cysteine proteases cathepsin B and L (CatB/L) and it was shown that blocking these proteases prevented infection. Another protein that is relevant to SARS-CoV-2 pathogenesis is angiotensin-converting enzyme 2 (ACE2), which plays a critical role in coronavirus cellular ingress. ACE2 expression levels are the highest in the heart, adipose tissue, small intestine, testis, kidneys, and thyroid. ACE2 is also expressed in the lungs, colon, liver, bladder, adrenal gland, blood, spleen, bone marrow, brain, blood vessels, and muscle. ACE2 is a type I transmembrane metallocarboxypeptidase which has been investigated by several independent researchers as the coronaviral cellular entry receptor and is also responsible for coronaviral attachment.

The first step of coronavirus entry process is the binding of the N-terminal portion of the viral protein unit S1 to a pocket of the ACE2 receptor. The second step, which is believed to be of utmost importance for viral entry, is the protein cleavage between the S1 and S2 units, which is operated by the receptor transmembrane protease serine 2 (TMPRSS2). The cleavage of the viral protein by TMPRSS2 is a crucial step because, after S1 detachment, the remaining viral S2 unit undergoes a conformational rearrangement which drives and completes the fusion between the viral and cellular membrane, with subsequent entry of the virus into the cell, release of its content, replication, cell lysis, and infection of other cells.

Venous thromboembolism has been associated with severe SARS-CoV-2 infection. Because ACE2 receptors limit vasoconstriction, inflammation, and thrombosis in the body, it has been postulated that the entry of SARS-CoV2 into the cells through membrane fusion markedly down-regulates ACE2 receptors, with loss of the catalytic effect of these receptors at the external site of the membrane. Increased pulmonary inflammation and coagulation have been reported as unwanted effects of the down regulation of ACE2 receptor.

Some patients may develop a syndrome known as pulmonary intravascular coagulation wherein immune-complexes activate intravascular coagulation that exist primarily in the pulmonary vasculature. Because patients with some types of viral infections can develop thromboses, these patients are often treated with varying types and levels of anticoagulation.

In certain aspects, the methods provided herein comprise treating a viral infection by administering a protease inhibitor optionally conjointly with an antiviral agent. Antiviral agents are pharmaceutical agents that can inhibit viral growth. Such agent may include but are not limited to penciclovir, acyclovir, famciclovir, valacyclovir, tenofovir disoproxil fumarate, lamivudine, zidovudine, didanosine, emtricitabine, stavudine, nevirapine, abacavir, raltegravir, dolutegravir, darunavir, ritonavir, cobicistat, efavirenz, ribavirin, neuraminidase inhibitor, recombinant interferons, recombinant immunoglobulins, oseltamivir, zanamivir, peramivir, baloxavir marboxil, laninamivir, remdesivir (RDV), tilorone, favipiravir, IFN-alpha, IFN-beta,1FN-gamma, IFN-lambda, peginterferon-alpha, peginterferon-beta, ribavirin, lopinavir/ritonavir, TAK888, adefovir, amantadine, rintatolimod (Ampligen), amprenavir, umifenovir (Arbidol), atazanavir, and ivermectin. Therapeutically effective amounts for treatment are familiar to those skilled in the art.

### Hematological and Pulmonary Conditions and Cardiac, Brain, and Reproductive Damage

The methods of the invention are useful for treating or preventing hematological and pulmonary conditions, and cardiac, brain, and reproductive damage. Hematological conditions are pathological conditions that primarily affect the blood & blood-forming organs. Pulmonary conditions are conditions that primarily affect the lungs.

In some embodiments, the pulmonary condition is hypoxemia. Hypoxemia refers to the low oxygen levels in the blood in a subject. The condition ultimately reduces oxygen throughout the body. Chronic hypoxemia symptoms include lung tightness, breathlessness, coughing, low lung capacity and volume. Hypoxemia can be caused by injury to the lungs, lung and sinus diseases, lung infections, lung cancer, and a host of medications that can injure lung cells and decrease the production of lung surfactants. Subjects with hypoxemia are usually on oxygen therapy.

Hypoxemia may be defined in terms of reduced partial pressure of oxygen (mm Hg) in arterial blood, but also in terms of reduced content of oxygen (ml oxygen per dl blood) or percentage saturation of hemoglobin (the oxygen binding protein within red blood cells) with oxygen. Acute hypoxemia can cause symptoms such as breathlessness or an increased rate of breathing. However, in a chronic context, and if the lungs are not well ventilated, hypoxemia can result in pulmonary hypertension, overloading the right ventricle of the heart and causing cor pulmonale and right sided heart failure. Severe hypoxemia can lead to respiratory failure. Many subjects with lung or sinus diseases or lung infections experience hypoxemia. The condition may be due to destruction of the alveoli in the lungs or the inadequate production of lung surfactants that enhance oxygen uptake.

In some embodiments, the hematological condition is disseminated intravascular coagulation (DIC). DIC is characterized by a systemic activation of the blood coagulation system, leading to subsequent clot formation, blood vessel obstruction and organ dysfunction. The large consumption of platelets and coagulation factors in this process may in turn cause bleeding, which further worsens the subject's condition and decreases the chances of survival. DIC is usually caused by an underlying condition in a subject, such as systemic inflammatory response syndrome (SIRS), sepsis, trauma, malignancy, heat stroke and hyperthermia. SIRS and sepsis are among the most common causes of DIC. Between 30% and 50% of sepsis patients develop DIC and sepsis severity positively correlates with DIC incidence and therefore mortality. Diagnosis of DIC is based on the clinical presentation of the underlying condition, along with abnormalities in laboratory parameters (prothrombin time, partial thromboplastin time, fibrin degradation products, D-dimer, or platelet count). The primary treatment of DIC is to address the underlying condition that is the responsible coagulation trigger. Blood product support in the form of red blood cells, platelets, fresh frozen plasma, and cryoprecipitate may be used to treat or prevent clinical complications.

In some embodiments, the pulmonary condition is pulmonary intravascular coagulopathy (PIC). Pulmonary intravascular coagulopathy is distinct from disseminated intravascular coagulation. PIC was first coined in McGonagle D, Sharif K, O'Regan A, Bridgewood C Autoimmun Rev. 2020 May 1; 102560 during the COVID-19 pandemic. Key features of COVID-19 related PIC are elevated levels of D-dimers and cardiac enzymes, pulmonary vascular bed thrombosis and fibrinolysis, and emergent pulmonary hypertension induced ventricular stress. Fibrinogen and C-reactive protein (CRP) levels were also both significantly elevated in PIC. CRP is a marker of inflammation, especially cardiac damage. High levels of CRP are correlated with high disease severity and mortality in COVID-19 patients. PIC is also characterized by hundreds of small blood clots throughout the lungs, which is typically not seen with other types of lung infections. The numerous blood clots are the cause of dramatically decreased blood oxygen levels in severe SARS-COV-2 infection.

In some embodiments, the hematological condition is septic shock. Septicaemia is an acute and serious bloodstream infection. Septicaemia occurs when a bacterial or viral infection elsewhere in the body, such as in the lungs or skin, enters the bloodstream. Entry of microbes into the blood stream is dangerous because the microbes and their toxins can be carried through the bloodstream to a subject's entire body. Septicaemia can quickly become life-threatening and it must be rapidly treated. If it is left untreated, septicaemia can progress to sepsis. Sepsis is a serious complication of septicaemia. Sepsis is when inflammation throughout the body occurs. This inflammation can cause blood clots and block oxygen from reaching vital organs, resulting in organ failure. When the inflammation occurs with extremely low blood pressure, septic shock occurs. Septic shock is fatal in many cases. Sepsis may manifest into sepsis-induced coagulopathy (SIC) or sepsis-associated coagulopathy (SAC). The International Society on Thrombosis and Hemostasis (ISTH) DIC and the Japanese Association for Acute Medicine (JAAM) DIC provide criteria on determining subject selection for SIC and SAC.

In some embodiments, the hematological condition is vascular leak syndrome. Vascular leak syndrome (VLS) is characterized by fever, hypotension, peripheral edema and hypoalbuminemia. VLS can occur as a side effect of illnesses due to pathogens such as viruses and bacteria. VLS is characterized by an increase in vascular permeability accompanied by extravasation of fluids and proteins resulting in interstitial edema and organ failure. Manifestations of VLS include fluid retention, increase in body weight, peripheral edema, pleural and pericardial effusions, ascites, anasarca and, in severe form, signs of pulmonary and cardiovascular failure. Symptoms are highly variable among patients and the causes are poorly understood. Endothelial cell modifications and/or damage are thought to be important in vascular leak. The pathogenesis of endothelial cell (EC) damage is complex and can involve activation or damage to ECs and leukocytes, release of cytokines and of inflammatory mediators, alteration in cell-cell and cell-matrix adhesion and in cytoskeleton function. Biomarkers to identify vascular leak syndrome include low albumin, elevated C-reactive protein (CRP), and elevated IL-6.

In certain aspects, the methods of the invention are useful for improving cardiac performance in a subject. Improved cardiac performance may be characterized, for example, by decreased pulmonary artery pressure and diminishing levels of CRP and/or troponin.

In certain aspects, the methods of the invention are useful for limiting the effects of neurovascular ischemia such as ischemic reperfusion injury that may be induced by intravascular coagulation.

In certain aspects, the methods of the invention are useful for limiting the effects of cardiovascular ischemia.

In certain aspects, the methods of the invention are useful for improving pulmonary function in a subject. Improved pulmonary performance may be characterized by decreased respiratory rate, rapid improvement of oxygation, suppression of fibrinolysis by euglobulin lysis activity, decreased levels of C-reactive protein (CRP), and/or rapid improvement on a 7-point ordinal scale. In some embodiments, the subject is free of respiratory failure. Respiratory failure is defined as the need for mechanical ventilation, extracorporeal membrane oxygenation (ECMO), non-invasive ventilation, or high flow oxygen devices.

In certain aspects, the methods of the invention are useful for treating or preventing cardiac damage.

In certain aspects, the methods of the invention are useful for treating or preventing brain damage.

In certain aspects, the methods of the invention are useful for treating or preventing reproductive damage.

### Cytokine Storm

The methods of the invention are useful for inhibiting cytokine storm. Cytokine storm (also known as hypercytokinemia) is a significant immune response to pathogens that invade the body. For example, the influenza A (H1N1) virus may trigger cytokine storms within the body. During a cytokine storm, pro-inflammatory mediators, such as Interleukin-1 (IL1), Interleukin-6 (IL6), tumor necrosis factor-alpha (TNF-alpha), oxygen free radicals, and coagulation factors are released by the immune cells.

Cytokine storms may also be associated with a number of non-infectious diseases, including adult respiratory distress syndrome (ARDS) and systemic inflammatory response syndrome (SIRS). Acute respiratory distress syndrome (ARDS) is a serious lung condition that causes low blood oxygen. Individuals who develop ARDS are usually ill due to another disease or a major injury. In ARDS, fluid builds up inside the tiny air sacs of the lungs, and surfactant breaks down. Surfactant is a foamy substance that keeps the lungs fully expanded so that a person can breathe. These changes prevent the lungs from filling properly with air and moving enough oxygen into the bloodstream and throughout the body. The lung tissue may scar and become stiff.

ARDS develops in response to lung damage due to underlying illnesses such as sepsis, pneumonia, COVID-19 or other issues. ARDS pathogenesis is mediated in part by proteinase-activated receptors (PAR 1-4). PAR 1 and PAR 2 play key roles in mediating the interplay between coagulation and inflammation and tissue repair and fibrosis. PARs are activated by proteases, including serine proteases that are inhibited by serine protease inhibitors such as nafamostat. Nafamostat is a potent inhibitor of PAR-activating proteases such as thrombin and tryptase.

Thrombin activates PAR-1 and PAR-4 in platelets. The downstream effects of PAR-1 include:
Disruption of adherens junctions between cells, increasing lung microvessel permeability
Upregulation of selectins and ICAM (adhesion molecules for neutrophils)
Release of inflammatory mediators:
   IL-1, IL-2, IL-6, IL-8, TNFα, CCL2
Downstream increase in Tissue Factor (TF) release, which further stimulates coagulation
   Tryptase activates PAR-2 on endothelial cells. Downstream effects of PAR-2 include:
Pro- or anti-inflammatory effects, depending on concentration and local conditions
Release of IL-8
Compromising barrier function and promoting sepsis and acute lung injury in animal models
Suppressing expression of Ve-cadherin
Inducing neutrophil and lung fibroblast migration
Inducing TF expression and von Willebrand factor release, promoting coagulation

Nafamostat inhibits human tryptase activity (e.g., IC₅₀ of 1.6×10⁻¹¹ M). Nafamostat inhibits thrombin activity in a potent, specific and reversible way (e.g., IC₅₀ values ranging from 1.9×10⁻⁶M to 3.3×10⁻⁷).

Non-limiting examples of ARDS biomarkers relevant to nafamostat's mechanism of action include endothelial damage markers, such as Ang-1, Ang-2, ICAM-1, selectins, VEGF, vWF, PA-1, protein C, and coagulation and fibrinolysis markers, such as PA-1, Protein C, thrombomodulin, Tissue Factor, and cell-free hemoglobin. Key PAR-dependent biomarkers in ARDS are listed in Table 1.

TABLE 1
Key PAR-dependent biomarkers in ARDS

| Marker | Function | Diagnostic or Prognostic |
|---|---|---|
| Endothelium damage | | |
| vWF | Secreted multimeric glycoprotein, | Prognostic |
| | marker of endothelial injury: acts | Increased vWF |
| | as a bridge for platelet adhesion, | is associated with |
| | and can promote platelet | an increased likelihood of |
| | aggregation | progression to ARDS |
| | | (debated), and |
| | | decreased survival |
| Selectins | Cell surface lectins that | Diagnostic and Prognostic |
| | mediate adhesion of leukocytes | Increased soluble plasma |
| | and platelets to endothelial cells | levels in ARDS are |
| | | associated with |
| | | decreased survival |
| ICAM | Soluble intercellular adhesion | Diagnostic |
| (P-selectin) | molecule on endothelia | Presence is associated |
| | | with increased likelihood |
| | | of progression |
| | | to ARDS |
| | | Prognostic |
| | | Increased soluble plasma |
| | | levels in ARDS, is |
| | | associated with decreased |
| | | survival, worse |
| | | outcomes |
| Coagulation and fibrinolysis | | |
| Tissue | Membrane-bound activator | Diagnostic |
| factor | of Factor VIIa, leading eventually | In ARDS, increased |
| | to thrombin formation and | levels of TF in lung, |
| | fibrin deposition | especially in |
| | | sepsis-induced ARDS |

SIRS is a serious condition related to systemic inflammation, organ dysfunction, and organ failure. It is a subset of cytokine storm, in which there is abnormal regulation of various cytokines. SIRS is also closely related to sepsis and subjects that satisfy criteria for SIRS may also have a suspected or proven infection. SIRS may be generally manifested as a combination of vital sign abnormalities including fever or hypothermia, tachycardia, tachypnea, and leukocytosis or leukopenia. SIRS is nonspecific and can be caused by ischemia, inflammation, trauma, burns, infection, pancreatitis, stress, organ injury, major surgery, fractures, or several insults combined. Thus, SIRS is not always related to infection.

Cytokine storms have the potential to cause significant damage to body tissues and organs. For example, occurrence of cytokine storms in the lungs can cause an accumulation of fluids and immune cells in the lungs and eventually block off the body's airways, thereby resulting in respiratory distress and even death. It has been suggested that pulmonary fibrosis is a potential consequence of severe SARS-CoV-2 infection. During SARS-CoV-2 infection, the immune response in the lungs is robust and, as a result, scar tissue called fibrosis forms. Pulmonary fibrosis is a condition that causes lung scarring and stiffness and impedes proper lung functioning.

### Anticoagulant Therapy

Blood clotting, also known as coagulation, is a process that is essential for the survival of mammals. The process of clotting can be divided into four phases. The first phase, vasoconstriction, decreases blood loss in the damaged area. In the next phase, platelet activation occurs by thrombin formation and the platelets attach to the site of the vessel wall damage forming a platelet aggregate. In the third phase, formation of clotting complexes leads to massive formation of thrombin, which converts soluble fibrinogen to fibrin by cleavage of two small peptides. In the fourth phase, after wound healing, the fibrin clot is dissolved by the action of the key enzyme of the endogenous fibrinolysis system called plasmin.

Two alternative pathways can lead to the formation of a fibrin clot in the coagulation cascade: the intrinsic and the extrinsic pathways. Both pathways comprise a relatively large number of proteins, which are known as clotting factors. The intrinsic and extrinsic pathways are initiated by different mechanisms, but converge to give a common final path of the clotting cascade. In this final path of clotting, clotting factor X is activated (Factor Xa) and is responsible for the formation of thrombin from the inactive precursor prothrombin circulating in the blood.

Venous thromboembolism (VTE) is a condition in which a blood clot forms most often in the deep veins of the leg, groin or arm (known as deep vein thrombosis, DVT) and travels through blood circulation, lodging in the lungs (known as pulmonary embolism, PE). Anticoagulants are effective at reducing the risk of blood clots caused by VTE. Anticoagulant therapy prevents blood clots by blocking specific coagulation factors in the coagulation cascade.

In certain aspects, provided herein is a method of treating a viral infection in a subject, comprising administering a protease inhibitor optionally conjointly with anticoagulant therapy. Exemplary anticoagulant therapies are listed in Table 2.

In some embodiments, the anticoagulant therapy is a heparin. Heparins are biologically active agents of the glycosaminoglycan family, extracted from natural sources, and have valuable anticoagulant and antithrombotic properties. The molecule has a negative charge density. Heparin is widely used as a clinical anticoagulant for such indications as cardiopulmonary bypass surgery, deep vein thrombosis, pulmonary thromboembolism, arterial thrombosis, and prophylaxis against thrombosis following surgery. Some forms of heparin have average molecular weights from 2 kDa to 30 kDa, such as between 12 kDa and 15 kDa. Heparin functions as an anticoagulant by indirectly inhibiting the enzymatic activity of factor Xa and thrombin through its ability to enhance the action of the plasma anticoagulant protein, antithrombin. Therapeutic plasma concentrations of heparin are generally 0.2-0.7 units/ml.

Heparin derivatives used in current clinical anticoagulation therapy include unfractionated heparin (UFH), low molecular weight heparin (LMWH), ultra-low molecular weight heparin (ULMWH) and the synthetic pentasaccharide derivatives fondaparunix and idraparinux. Low molecular weight embodiments of heparin include enoxaparin, dalteparin, fondaparinux, tinzaparin, certoparin, ardeparin, nadroparin, parnaparin, and reviparin and ultra-low molecular weight embodiments of heparin include semuloparin. Low MW heparins act primarily as factor Xa inhibitors, as they enhance antithrombin's anticoagulant effect toward factor Xa to a much greater extent than toward thrombin. Low MW heparins are widely used for longer-term anticoagulant therapy to prevent deep vein thrombosis and have certain advantages over unfractionated heparin. Therapeutic plasma concentrations of low MW heparins are generally 0.2-2 units/ml. Low MW heparins have plasma half-lives of 4-13 hours, resulting in prolonged anticoagulation even if the drug is discontinued when bleeding occurs.

In some embodiments, the anticoagulant therapy is argatroban. Argatroban is an oral anticoagulant drug that is approved for patients at risk for thrombosis who cannot be treated with heparin. Therapeutic plasma concentrations are about 1μg/ml. As a derivative of L-arginine, argatroban is a competitive inhibitor of thrombin and only interacts with the active site of thrombin. It directly inactivates the activity of thrombin (clotting factor IIa) and has no direct action on the generation of thrombin. The function of argatroban is independent of the anti-thrombin in the body. Argatroban inactivates not only thrombin in free state in blood, but also inactivates the thrombin combined with fibrin thrombus, blocks the positive feedback of the coagulation cascade, and inhibits thrombin-induced platelet aggregation even in a very low concentration, which indirectly inhibits the formation of thrombin. Due to its small molecular weight, argatroban can enter the inside of a thrombus, directly inactivate the thrombin already combined with fibrin thrombus, and even exhibits an antithrombotic effect against an early-formed thrombosis. Furthermore, argatroban can greatly decrease the level of thrombin-antithrombin complex (TAT) in plasma, effectively reduce the hypercoagulable state of patients, and has very good clinical results in treating chronic thromboembolic disease.

In some embodiments, the anticoagulant therapy is dabigatran. Dabigatran is a potent, reversible, monovalent direct thrombin inhibitor. Dabigatran reduces the risk of stroke and systemic embolism in patients with non-valve atrial fibrillation. It is also useful in the primary prophylaxis of venous thromboembolic complications in adult patients who underwent surgery for elective total hip arthroplasty or surgery for total knee arthroplasty. Dabigatran inhibits free thrombin, fibrin-linked thrombin, and thrombin-induced platelet aggregation. Dabigatran was first disclosed in WO 1998/37075 (incorporated herein by reference in its entirety), which claims compounds with a thrombin inhibiting and thrombin prolonging action, called 1-methyl-2-[N-[4-(N-n-hexyloxycarbonylamidino) phenyl] aminomethyl] benzimidazol-5-ylcarboxylic acid-N-(2-pyridyl)-N-(2-ethoxycarbonylethyl) amides.

In some embodiments, the anticoagulant therapy is rivaroxaban. Rivaroxaban is an anticoagulant compound 5-chloro-N-{[(5S)-2-oxo-3-[4-(3-oxomorpholin-4-yl) phenyl] oxazolidin-5-yl] methyl} thiophene-2-carboxamide, which was originally disclosed in WO 2001/47919 A1 (incorporated herein by reference in its entirety). Rivaroxaban is a small molecule inhibitor of blood clotting factor Xa. and is used in the prevention and treatment of thromboembolic diseases such as myocardial infarction, angina pectoris, reocclusion and restenosis after angioplasty or shunt, stroke, stroke transient ischemic, peripheral arterial obstructive diseases, pulmonary embolism and venous thrombosis.

In some embodiments, the anticoagulant therapy is apixaban. Apixaban and its method of manufacture are described in U.S. Pat. Nos. 6,967,208 and 7,396,932, and PCT publications WO 2007/001385 and WO2006/13542, the disclosures of which are incorporated herein by reference in its entirety.

In some embodiments, the anticoagulant therapy is edoxaban. Edoxaban is disclosed in WO 2013/026553, incorporated herein by reference in its entirety. Edoxaban is a member of the so-called “Xaban-group” and is a low molecular inhibitor of the enzyme factor Xa, which participates in the blood coagulation system. Therefore, edoxaban is classified as an antithrombotic drug and its possible medical indications are reported to be treatment of thrombosis and thrombosis prophylaxis after orthopedic operations, such as total hip replacement, as well as for stroke prevention in patients with atrial fibrillation, the prophylaxis of the acute coronary syndrome and the prophylaxis after thrombosis and pulmonary embolism.

In some embodiments, the anticoagulant therapy is enoxaparin.

In some embodiments, the anticoagulant therapy is ardeparin.

In some embodiments, the anticoagulant therapy is tinzaparin.

In some embodiments, the anticoagulant therapy is dalteparin.

In some embodiments, the anticoagulant therapy is fondaparinux. In some embodiments, the anticoagulant therapy is administered at VTE Prophylaxis Dose. In some embodiments, the anticoagulant therapy is administered at standard dose. In some embodiments, the anticoagulant therapy is administered at intermediate dose. In some embodiments, the anticoagulant therapy is administered at full dose. Dosing amounts of anticoagulant therapies are listed in Table 3. For purposes of disclosure, VTE prophylaxis standard anticoagulant dose, intermediate anticoagulant dose, and full anticoagulant dose are specific to the indicated anticoagulant medications.

TABLE 2
Exemplary Anticoagulant Therapies

| Drug | Chemical Structure |
|---|---|
| Heparin | 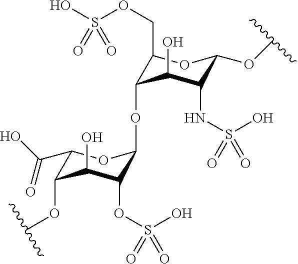 |
| Enoxaparin | 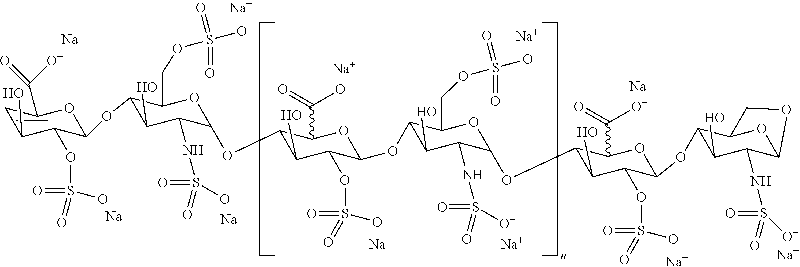 |
| Ardeparin | 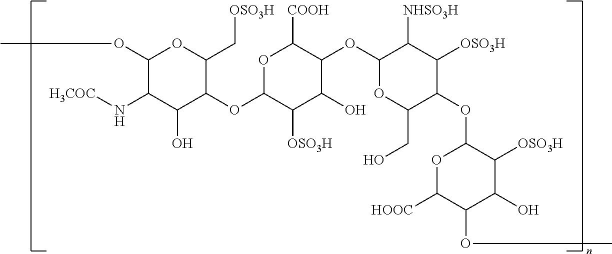 |
| Tinzaparin | 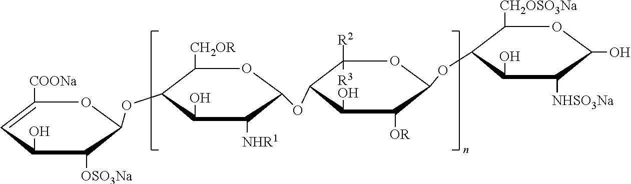 |
| Dalteparin | 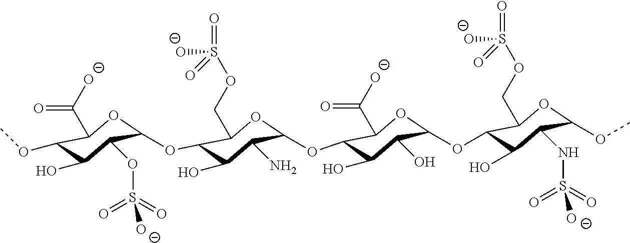 |
| Fondaparinux | 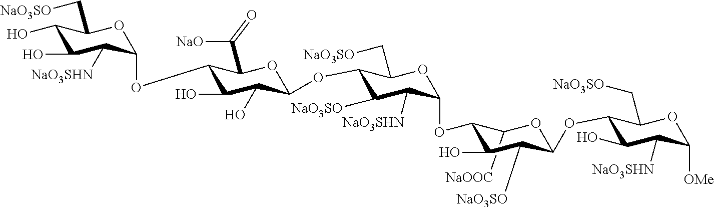 |
| Argatroban | 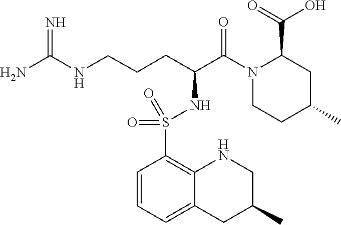 [SMILES: C[C@@H]1CNc2c(cccc2S(=O)(=O)N[C@@H](CCCNC(=N)N)C(=O)N2CC[C@@H](C)C[C@@H]2C(=O)O)C1] |
| Apixaban | 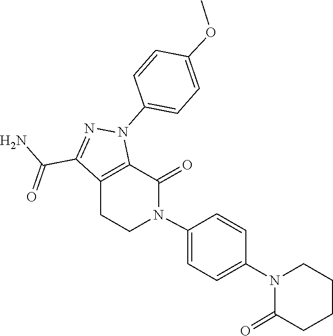 [SMILES: COc1ccc(-n2nc(C(N)=O)c3c2C(=O)N(c2ccc(N4CCCCC4=O)cc2)CC3)cc1] |
| Dabigatran | 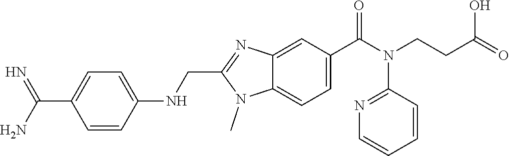 [SMILES: Cn1c(CNc2ccc(C(=N)N)cc2)nc2cc(C(=O)N(CCC(=O)O)c3ccccn3)ccc21] |
| Rivaroxaban | 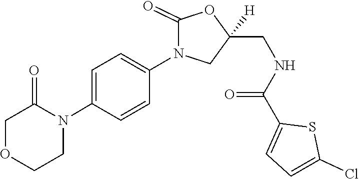 [SMILES: O=C(NC[C@H]1CN(c2ccc(N3CCOCC3=O)cc2)C(=O)O1)c1ccc(Cl)s1] |
| Edoxaban | 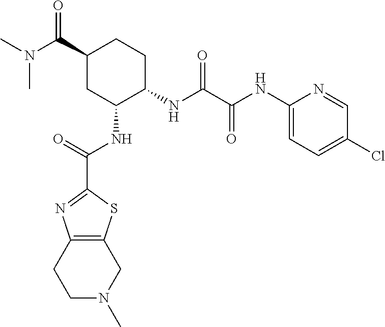 [SMILES: CN1CCc2nc(C(=O)N[C@@H]3C[C@@H](C(=O)N(C)C)CC[C@@H]3NC(=O)C(=O)Nc3ccc(Cl)cn3)sc2C1] |

TABLE 3
Exemplary Anticoagulant Therapy Doses

| | VTE | | |
|---|---|---|---|
| | Prophylaxis | Intermediate | Full Anticoagulant |
| Drug | Standard Dose | Anticoagulant Dose | Dose |
| Heparin | 10,000-20,000 | Dose > 20,000 units | Dose > 20,000 |
| | units/day | per day and aPTT less | units per day |
| | | than 60 seconds | and aPTT |
| | | | greater than 60 |
| | | | seconds |
| Enoxaparin | 30-50 mg daily | | 1 mg/kg twice |
| Lovenox | | | daily Or |
| | | | 1.5 mg daily |
| Dalteparin | 2,500-5,000 | | 200 IU/kg total |
| | IU daily | | body weight |
| | | | subcutaneously |
| | | | once daily. |
| Fondaparinux | 2.5 mg daily | | Daily Dose: |
| | | | 5 mg (body |
| | | | weight < 50 kg), |
| | | | 7.5 mg (body |
| | | | weight 50 to |
| | | | 100 kg), or |
| | | | 10 mg (body |
| | | | weight > 100 kg) |
| Argatroban | N/A | 0.1-0.5 mcg/kg/min | 0.5-1.0 mcg/kg/min |
| Apixaban | 2.5-5.0 mg | | 10-20 mg daily |
| | daily | | |
| Dabigatran | 200 mg | | 300 mg daily |
| | daily | | (150 mg daily in |
| | | | patients with |
| | | | reduced CrCl) |
| rivaroxaban | 10 mg daily | | 15-20 mg daily |
| Edoxaban | | | 30-60 mg daily |

### Protease Inhibitors

A protease is an enzymatic protein which acts to cleave peptide bonds on proteins. There are several types of proteases, often named for the amino acid target where the cleavage event occurs (serine, threonine, or tyrosine). Certain cleavage events can activate specific cell receptors leading to activation of protein kinases and downstream intracellular signaling pathways. Protein kinases are enzymes which perform the catalyzation of phosphorylation action to amino acids. Downstream effects of kinase activation may include cytokine activation. Cytokines are proteins responsible for cellular signaling and regulatory functions within the body.

Proteases and protein kinases play a vital role in the infection mechanisms for viruses. During some virus infections, like Severe Acute Respiratory Syndrome (SARS) or Dengue, cellular damage triggers kinase activation resulting in inflammation. One such kinase, Protein Kinase R (PKR, a serine/threonine kinase) is activated by proteolytic cleavage and by cytokines type I interferons (IFN-α and β). This activation in turn can result in apoptosis of the cell through action by eukaryotic translation initiation factor 2 (eIF2α), which may be important in virus replication since it is responsible for regulating mRNA translation.

Other kinases may be activated by coronavirus proteolytic events, which have the ability to facilitate autophosphorylation of eIF2a such as PKR-like endoplasmic reticulum kinase (PERK) and general control nonderepressible-2 kinase (GCN2). There are also avenues where PKR activation may lead to apoptosis without activation of eIF2α, and there is evidence that kinases other than PKR are involved in the eIF2α phosphorylation, which is hypothesized to facilitate the coronavirus infection cycle. For influenza viral infection, important signaling pathways are nuclear factor (NF-κB) signaling, PI3K/Akt pathway, MAPK pathway, PKC/PKR signaling, and TLR/RIG-I signaling cascades, all of which are facilitated by protein kinase activities that are activated by proteases. The kinase activation that occurs with these virus infections can result in organ damage from the resulting inflammation and coagulation.

Protease inhibitors are molecules which can interfere with the mechanisms of cell signaling through prevention of kinase activation. The medical and scientific community understand the opportunity for management of diseases through modulation of protein signaling through kinases and a growing body of knowledge indicates that protease inhibitors may provide specific utility in the management of virus infections such as coronavirus, influenza virus, Dengue, etc. and may provide some relief to the inflammation symptomology of virus infections. Currently, there are several protease inhibitors (PI) that have been used or investigated clinically to treat a variety of patient populations including those with pancreatitis, chronic obstructive pulmonary disease (COPD), cancer, arthritis, hypertension, and those in need of anticoagulation. In addition, some proteases are an effective treatment option for treating patients with viral infections.

Often, viral infection relies on the proteolytic activation of host proteins as part of the cellular ingress mechanism. The human protease TMPRSS2 is the primary target for the protease inhibitor of the invention due to its importance in SARS-CoV-2 virus infection and pathogenesis. Coronavirus is not the only agent which may be subject to treatment with protease inhibitors. Other viruses like West Nile and Dengue have been investigated for treatment using other protease inhibitors which help prevent infection by the viruses, but also show an alteration of the inflammatory cytokine response.

The reduction of the cytokine response may be clinically important as many patients suffer immunomodulated impacts of the virus infection through the “kinase cascade” resulting in enhanced morbidity and mortality with some viruses. Protein kinases are responsible for signaling pathways regulating inflammation which may be exacerbated by viral triggered, pro-inflammatory cytokine storms which damage organ tissue.

In certain aspects, provided herein are methods for treating a viral infection comprising administering a protease inhibitor. In some embodiments, the protease inhibitor is optionally administered conjointly with an anticoagulation therapy. In some embodiments, the protease inhibitor is administered at a starting dose of 5 mcg/kg/hour, 10 mcg/kg/hour, 25 mcg/kg/hour, 50 mcg/kg/hour, 75 mcg/kg/hour, 100 mcg/kg/hour, 125 mcg/kg/hour, 150 mcg/kg/hour, 175 mcg/kg/hour, or 200 mcg/kg/hour. In some embodiments, the protease inhibitor is administered at a maintenance dose 10 mcg/kg/hour, 50 mcg/kg/hour, 100 mcg/kg/hour, 150 mcg/kg/hour, 200 mcg/kg/hour, 250 mcg/kg/hour, 300 mcg/kg/hour, 350 mcg/kg/hour, 400 mcg/kg/hour, 450 mcg/kg/hour, 500 mcg/kg/hour, 550 mcg/kg/hour, 600 mcg/kg/hour, 650 mcg/kg/hour, 700 mcg/kg/hour.

In some embodiments, the protease inhibitor is administered at a starting dose for over 2 hours, 3 hours, 4 hours, 5 hours, 6 hours, 7 hours, 8 hours, or 9 hours.

In some embodiments, the protease inhibitor is administered at a maintenance dose for up to 7 days, 9 days, 11 days, 13 days, 15 days, 17 days, 19 days, or 21 days.

In some embodiments, administration of the protease inhibitor reduces viremia in a subject. In some embodiments, administration of the protease inhibitor results in clearance of the viral infection. Viral clearance may be determined by resolution of viral infection symptoms.

In some embodiments, the protease inhibitor is a serine protease inhibitor. Exemplary serine protease inhibitors are listed in Table 3.

TABLE 3
Exemplary Serine Protease Inhibitors

| Serine Protease | |
|---|---|
| Inhibitor | Chemical Structure |
| Camostat mesylate | 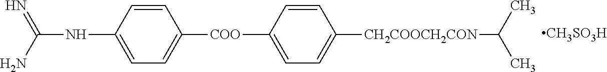 |
| Nafamostat mesylate | 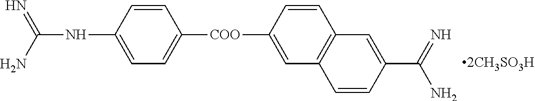 |
| Gabexate mesylate | 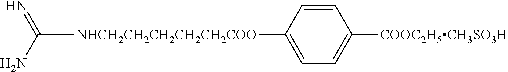 |

In some aspects, the protease inhibitor (or the protease inhibitor agent) used by the disclosed methods is nafamostat, nafamostat mesylate, or another salt form of nafamostat. For any embodiment disclosed herein in which a molecule (such as nafamostat) is used pharmacologically, the salt form can be a pharmaceutically acceptable salt form. Nafamostat is a small molecule, broad spectrum, protease inhibitor that inhibits thrombin at the platelet thrombin receptor, PAR1.

Nafamostat is a potent, synthetic, small molecule, broad spectrum, serine protease inhibitor.

Nafamostat is efficacious due to its inhibition of a broad spectrum of serine proteases involved in a variety of signaling pathways involved in inflammation and reverse vascular leakage which are also involved in facilitating virus infection and propagation.

Nafamostat is potentially efficacious due to its function as an anticoagulation agent which has been shown to have positive outcomes for those patients which have received personalized heparinization during treatment for disseminated intravascular coagulation (DIC).

Nafamostat inhibits virus protein targets like Coronavirus S-Protein, and human cellular proteins involved in virus infection pathways, particularly serine protease TMPRSS2, which is critical for viral spread and pathogenesis in an infected host. Additional inhibited targets are NF-κB and endosomal protein cathepsin B.

Nafamostat is capable of inhibiting pro-inflammatory cytokines typically associated with the “cytokine storm” such as interleukin-6 (IL-6) and interleukin-8 (IL-8). Nafamostat can also inhibit proteases within the VIIa complex. Nafamostat inhibits thrombin production, as well as the proteases human Hageman factor, prothrombin, trypsin-1, and kallikrein-1. Nafamostat has been shown to provide broad spectrum inhibition to the coagulation-fibrinolysis system (thrombin, XIIa, Xa, VIIa, and plasmin), the kallikrein-kinin system (kallikrein), the complement system (Clr, Cls, B, D) and pancreatic enzymes (trypsin, pancreatic kallikrein).

Nafamostat has a strong inhibitory action on the coagulation-fibrinolysis system (XIIa, Xa, VIIa, and plasmin), the kallikrein-kinin system (kallikrein), the complement system (Clr, Cls, B, D) and pancreatic enzymes (trypsin, pancreatic kallikrein) in vitro.

Platelet activation and aggregation occurs when the patient enters a proinflammatory state. The unique ability of nafamostat to inhibit platelet aggregation and disaggregate platelets in normal human platelet rich plasma may provide an important basis to support the clinical use of nafamostat in prolonging the life of a patient with complications such as platelet thrombosis.

### Methods of Delivering a Protease Inhibitor Agent to a Subject

Contemporary methods exist to deliver protease inhibitors to a patient including intravenous, subcutaneous, inhalation and oral routes of administration. In the case of suspected viral infection, in advance of the onset of symptoms, a patient could be provided oral dosage of a protease inhibitor to inhibit inflammation onset and potentially interfere with the virus infection pathway. For onset of symptoms, before or after development of inflammation, a patient could be delivered protease inhibitors subcutaneously into the lymph for delivery of the therapy to the sites of infection throughout the body. For onset of symptoms, but before severe inflammation, a patient could be delivered inhalation/intranasal protease inhibitors for direct delivery of the therapy to the sites of infection. For advanced condition, where lung function is reduced, and inflammation is severe, the patient could be infused as a part of the standard of care through intravenous application of protease inhibitors to the bloodstream.

In some embodiments, before delivering a protease inhibitor agent to a subject, the subject (or a subpopulation of subjects) can be selected as described in the next section.

In some embodiments, the protease inhibitor agent is administered as a single dose.

In some embodiments, the protease inhibitor agent is administered as a continuous dose.

In some embodiments, the protease inhibitor agent is administered from a depot via a microneedle array. In some embodiments, the protease inhibitor agent is administered by slow infusion. In some embodiments, the protease inhibitor agent is administered over about 5 days. In some embodiments, the protease inhibitor agent is administered subcutaneously.

### Methods of Selecting Subjects for Delivery of a Protease Inhibitor Agent

In some aspects, the disclosure relates to methods of selecting a subject, or a subpopulation of subjects, to whom a protease inhibitor agent will be delivered from an extracorporeal circuit. These methods include determining that a subject meets certain criteria and then selecting the subject for delivering a protease inhibitor agent from an extracorporeal circuit to the subject. The set of criteria that can be used include the following: (1) criteria for sepsis-induced coagulopathy; (2) criteria for sepsis-associated coagulopathy; (3) criteria of the ISTH for DIC; (4) criteria of the JAAM for DIC; (5) having a plasma procalcitonin level above its healthy reference range; (6) having a plasma nucleosome level above its healthy reference range; and (7) having a plasma syndecan-1 level and/or D-dimer level or CRP level above its healthy reference range. In some embodiments, the criteria include those enumerated as (1) through (4). In some embodiments, the criteria also include at least one, at least two, or all three enumerated as (5) through (7).

In certain aspects, the method of selecting subjects relates to selecting a subpopulation of subjects that have DIC. Diagnosing DIC facilitates sepsis management and is associated with improved outcomes. Although the ISTH has proposed criteria for diagnosing overt DIC, these criteria are not suitable for early detection. Moreover, no single biomarker can effectively diagnose DIC in patients with sepsis. The methods disclosed herein address these problems by identifying subjects that will ultimately benefit from treatment with a protease inhibitor agent.

In certain aspects, the method of selecting subjects relates to selecting a subpopulation of subjects that have PIC. Diagnosing PIC facilitates management of infection and intravascular thromboses. The hallmarks of PIC are increased D-dimer in the background of an infection. Patients with PIC typically have normal fibrinogen levels with elevated D-dimer. They may show signs and symptoms of macrophage activation syndrome (MAS) with features of hemophagocytosis, elevated hepcidin levels, and hypoferremia.

A biomarker that can be used for subject selection is procalcitonin (PCT). PCT levels can be significantly elevated in patients with sepsis and DIC compared to healthy controls. Interestingly, PCT levels can also be significantly higher in patients with DIC as compared to patients with sepsis without substantial coagulopathy. This suggests that plasma PCT levels may be a useful prognostic marker in detecting not only sepsis but also the progression of onset to DIC. Additionally, PCT may have a potential role in early risk stratification and prediction of overall morbidity and mortality.

Another biomarker that can be used for subject selection is D-dimer. D-dimer is a fibrin degradation product that is present in the blood after a blood clot is degraded by fibrinolysis. It contains two D fragments of the fibrin protein joined by a cross-link. D-dimer concentration may be determined in a subject by a blood test. Reference ranges for D-dimer in non-pregnant adults is less than or equal to 287 ng/mL. D-dimer is associated with the fragmentation of fibrin in coagulopathy and is currently used to identify pulmonary embolisms.

A biomarker that can be used for subject selection is Interleukin-6 (IL-6). IL-6 is a major pro-inflammatory cytokine and consists of 212 amino acids with two N-linked glycosylation sites. IL-6 signaling is mediated by the binding of IL-6 to either soluble or surface bound IL-6 receptor chain (IL-6R), enabling interaction of the complex with the cell surface transmembrane gp130 subunit. The interaction mediates intracellular signaling and is responsible for the proliferation and differentiation of immune cells. IL-6 plays a crucial role in coagulation; it is primarily involved in the up-regulation of tissue factors that initiate coagulation. IL-6 is also one of the major cytokines that is released from the lung in response to a wide variety of inflammatory stimuli during pulmonary intravascular coagulation. IL-6 can be measured in serum of the subject using methods well known in the art.

Another biomarker that can be used for subject selection is C-reactive protein (CRP). CRP is a pentraxin family member and is secreted by the liver. CRP may increase in response to either acute or chronic inflammation. CRP levels increase in response to macrophage and adipocyte secretion of IL-6 and lead to activation of the complement pathway. CRP levels may increase in response to microbial infection, inflammation, and tissue damage. As an acute-phase protein, levels of CRP can rise rapidly upon inflammation and thus CRP can function as a biomarker of active inflammation. Moreover, CRP can have a relatively short half-life and thus can also be used to monitor resolution of the inflammatory insult. CRP can be measured in the blood using a high-sensitivity C-reactive protein (hs-CRP) test.

Another biomarker that can be used for subject selection is a nucleosome level. Nucleosome levels can be significantly elevated in patients with overt DIC compared to healthy controls and compared to septic patients without DIC. This specific elevation of nucleosomes in patients with severe coagulopathy suggests that nucleosome level may be useful as a tool to identify patients with sepsis having overt DIC from patients with sepsis without coagulopathy.

Another biomarker that can be used for subject selection is syndecan-1. One of the pathophysiological processes in sepsis is endothelial dysfunction, which leads to DIC. Syndecan-1 is a major structural component of the endothelium and plays a key role in endothelial function. Syndecan-1 levels can be associated with not only the severity of illness and mortality but also DIC development in sepsis, suggesting that syndecan-1 could be a predictive marker of DIC. In patients with sepsis, syndecan-1 can correlate with the DIC score and can have strong discriminative power for the prediction of DIC development. This suggests that syndecan-1 could be a predictive marker of DIC in patients with sepsis.

Another biomarker that can be used for subject selection is von Willebrand factor (vWF). vWF is a secreted multimeric glycoprotein that acts as a bridge for platelet adhesion, and can promote platelet aggregation. It is a marker of endothelial injury, and in ARDS, increased vWF may be associated with an increased likelihood of progression to ARDS, and decreased survival. This suggests that vWF could be a predictive marker of ARDS progression in patients with sepsis, pneumonia, COVID-19, or other conditions that lead to ARDS, targeting those patients for early intervention with protease inhibitor treatment.

Another set of biomarkers that can be used for subject selection are selectins. Selectins are cell surface lectins (ICAM, a P-selectin, is one example) that mediate adhesion of leukocytes and platelets to endothelial cells, and increased soluble plasma levels of selectins are associated with increased likelihood of progression to ARDS and decreased survival. This suggests that soluble levels of selectins could be a predictive marker of ARDS progression in patients with sepsis, pneumonia, COVID-19, or other conditions that lead to ARDS, targeting those patients for early intervention with protease inhibitor treatment.

Another biomarker that can be used for subject selection is Tissue factor (TF). TF is a membrane-bound activator of Factor VIIa, leading eventually to thrombin formation and fibrin deposition in the fibroproliferative phase of ARDS. Increased levels of TF indicate ARDS, especially in sepsis-induced ARDS. TF could be a predictive marker of ARDS progression in patients with sepsis, pneumonia, COVID-19, or other conditions that lead to ARDS, targeting those patients for early intervention with protease inhibitor treatment.

Beyond reliance on biomarkers, other criteria can also be used to classify subjects. Two such different criteria for evaluating coagulopathy in sepsis include the following: sepsis-induced coagulopathy (SIC) and sepsis-associated coagulopathy (SAC). Although both use universal hemostatic markers of platelet count and pro-thrombin time, significance and usefulness of these criteria remain unclear. Additional criteria include the following: ISTH DIC criteria and the JAAM DIC criteria.

Coagulation factors and anticoagulant proteins not only play a role in hemostatic activation, but also interact with specific cell receptors leading to activation of signaling pathways. Specifically, protease interactions that modulate inflammatory processes may be important in sepsis. The most significant pathways by which coagulation factors regulate inflammation is by binding to protease-activated receptors (PARs). PARs are transmembrane G-protein coupled receptors and four different types (PAR 1-4) have been recognized. A typical property of PARs is that they serve as their own ligand. Proteolytic cleavage by an activated coagulation factor leads to exposure of a neoamino terminus, which is capable of activating the same receptor (and presumably adjacent receptors), leading to transmembrane signaling. PARs 1, 3, and 4 are receptors that are activated by thrombin while PAR-2 is triggered by the tissue factor-factor VIIa complex, factor Xa, and trypsin. PAR-1 is also a receptor for the tissue factor-factor VIIa complex and factor Xa.

### EXAMPLES

### Example 1: Protease Inhibitor Dosing

The use of nafamostat in COVID-19 and potential other anticoagulants requires a starting dose to ensure there is no interaction between the nafamostat and another medication utilized in COVID treatment. Table 4 below outlines the starting dose and maintenance dose of nafamostat for patients receiving no VTE prophylaxis, VTE prophylaxis specific to the drug and dose, and the dose of nafamostat for patients receiving intermediate and full anticoagulation. Table 3 (above) outlines the various drugs utilized for VTE prophylaxis and full dose anticoagulation and that corresponding level.

TABLE 4
Nafamostat Dosing

| | | | | Patient already |
|---|---|---|---|---|
| | | | | on Full |
| | Absent of | VTE | | Anticoagulant |
| | VTE | Prophylaxis | Intermediate | Dose that |
| Starting | Prophylaxis | at Standard | Anticoagulant | is not from |
| Dose | Dose | Dose | Dose | nafamostat |
| Nafamostat | 45-100 | 25-100 | 10-50 | 10-25 |
| (cont. | mcg/kg/hour | mcg/kg/hour | mcg/kg/hour | mcg/kg/hour |
| infusion) | over 2 hours | over 2 hours | over 2 hours | over 2 hours |
| Maintenance | 90-600 | 50-500 | 20-450 | 15-350 |
| Dose | mcg/kg/hour | mcg/kg/hour | mcg/kg/hour | mcg/kg/hour |
| (cont. | for up to | for up to | for up to 21 | for up to 21 |
| infusion) | 21 days | 21 days | days | days |

### Example 2: Example Nafamostat Composition

Nafamostat can be infused as a sterile solution containing the following ingredients listed in Table 5. A reconstitution solvent such as water or saline solution may be used to dilute to the desired infusion concentration.

TABLE 5
Sample Product Vial Contents (100 mg vial)

| | Amount | |
|---|---|---|
| Component | (mg) | Purpose |
| Mannitol | 200 | Mannitol improves the appearance and dissolution |
| | | characteristics of the lyophilized powder. |
| Nafamostat | 100 | Nafamostat is the active ingredient. |
| mesylate | | |
| Succinic acid | 10 | Succinic acid decreases the pH of the |
| | | solution, which improves the stability in water. |
| | | A lower pH solution is more stable when |
| | | dissolved with the recommended |
| | | diluent, 5% dextrose. |

Nafamostat may also be prepared as a non-aqueous solution in DMSO, which eliminates the need for lyophilization.

### INCORPORATION BY REFERENCE

Each publication and patent mentioned herein is hereby incorporated by reference in its entirety. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the following claims. The full scope of the invention should be determined by reference to the claims, along with their full scope of equivalents, and the specification, along with such variations.

1. A method of treating a viral infection, hypoxemia, pulmonary intravascular coagulopathy, disseminated intravascular coagulation, viremia, septic shock, neurovascular ischemia, cardiovascular ischemia, systemic inflammatory response syndrome (SIRS), acute respiratory distress syndrome (ARDS), edema, stroke, and/or vascular leak syndrome in a subject, comprising administering a protease inhibitor at a starting dose of 5-200 mcg/kg/hour, optionally conjointly with an anticoagulant therapy and/or an antiviral agent.

2. The method of claim 1, wherein the anticoagulant therapy is venous thromboembolism (VTE) prophylaxis.

3-4. (canceled)

5. The method of claim 2, wherein the protease inhibitor is administered with VTE prophylaxis at standard dose.

6. (canceled)

7. The method of claim 1, wherein the protease inhibitor is administered with the anticoagulant therapy at intermediate anticoagulant dose.

8. (canceled)

9. The method of claim 1, wherein the protease inhibitor is administered with the anticoagulant therapy at full anticoagulant dose.

10. (canceled)

11. The method of claim 1, wherein the protease inhibitor is administered for over 2 hours.

12. A method of treating a viral infection, hypoxemia, pulmonary intravascular coagulopathy, disseminated intravascular coagulation, viremia, septic shock, neurovascular ischemia, cardiovascular ischemia, systemic inflammatory response syndrome (SIRS), acute respiratory distress syndrome (ARDS), edema, stroke, and/or vascular leak syndrome in a subject, comprising administering a protease inhibitor at a maintenance dose of 10-700 mcg/kg/hour, optionally conjointly with an anticoagulant therapy and/or an antiviral agent.

13. The method of claim 12, wherein the anticoagulant therapy is venous thromboembolism (VTE) prophylaxis.

14-15. (canceled)

16. The method of claim 13, wherein the protease inhibitor is administered with VTE prophylaxis at standard dose.

17. (canceled)

18. The method of claim 12, wherein the protease inhibitor is administered with the anticoagulant therapy at intermediate anticoagulant dose.

19. (canceled)

20. The method of claim 12, wherein the protease inhibitor is administered with the anticoagulant therapy at full anticoagulant dose.

21. (canceled)

22. The method of claim 12, wherein the protease inhibitor is administered for up to 21 days.

23. The method of claim 1, wherein the protease inhibitor is nafamostat mesylate, camostat mesylate, aprotinin, or gabexate mesylate.

24. The method of claim 1, wherein the anticoagulant therapy is heparin, enoxaparin, ardiparin, tinzaparin, dalteparin, fondaparinux, argatroban, apixaban, dabigatran, rivaroxaban, laninamivir or edoxaban therapy.

25. The method of claim 1, wherein the viral infection is coronavirus, influenza, Dengue, West Nile, and/or a respiratory syncytial viral infection.

26-33. (canceled)

34. The method of claim 1, wherein administration of the protease inhibitor modulates a level of a biomarker of inflammation in the subject.

35. The method of claim 34, wherein the biomarker of inflammation is D-dimer, C-reactive protein (CRP), or IL-6.

36-37. (canceled)

38. The method of claim 1, wherein administration of the protease inhibitor inhibits cytokine storm in the subject, reduces inflammation in the subject, reduces platelet aggregation in the subject, improves cardiac performance in the subject, and/or improves pulmonary function in the subject.

39-43. (canceled)

44. A method of treating acute respiratory distress syndrome (ARDS) with a protease inhibitor in a subject in need thereof, comprising:
(a) determining whether the ARDS is characterized by a level of a biomarker of inflammation, a coagulopathy biomarker, an endothelial damage marker, and/or a coagulation and fibrinolysis marker above a threshold level; and
(b) if the ARDS is characterized by a level of a biomarker of inflammation, a coagulopathy biomarker, an endothelial damage marker, and/or a coagulation and fibrinolysis marker above the threshold level, administering a protease inhibitor to the subject.

45. The method of claim 44, wherein the endothelial damage marker is Ang-1 and/or Ang-2, ICAM-1, selectin, VEGF, vWF, PA-1, or protein C.

46-51. (canceled)

52. The method of claim 44, wherein the coagulation and fibrinolysis marker is PA-1, protein C, thrombomodulin, Tissue Factor, or cell-free hemoglobin.

53-63. (canceled)
